(19) United States
(12) Patent Application Publication
(10) Pub. No.: US 2003/0229274 A1
(43) Pub. Date: Dec. 11, 2003

(54) Electromyograph having telemetry

(75) Inventor: John W. Standeven, Rock Hill, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Barnes-Jewish Hospital

(21) Appl. No.: 10/456,331

(22) Filed: Jun. 6, 2003

Related U.S. Application Data
(60) Provisional application No. 60/386,841, filed on Jun. 7, 2002.

(51) Int. Cl. A61B 5/04

(52) U.S. Cl. 600/300; 600/546

(57) ABSTRACT

A system for measuring electrical signals produced by tissue of a patient. The system includes electrodes adapted for attachment to selected sites on the patient corresponding to tissue of interest and transmitters. Each transmitter is connected to at least one of the electrodes and adapted to transmit a signal at a telecommunications frequency uniquely selected to correspond to the electrode to which the transmitter is connected. The system also includes a receiver configured to receive at least one signal transmitted by the transmitters at the uniquely selected telecommunications frequency.

### TECHNICAL FIELD

The present invention relates generally to telemetry, and more specifically to a method and apparatus for telemetering a signal representing a physical response.

### BACKGROUND OF THE INVENTION

Analysis of walking is often done in a “Gait Lab.” One portion of the analysis includes measuring and analyzing the leg muscles. Muscles generate electrical signals during movement. These muscle signals are recorded on an electromyogram which may be measured and analyzed. Prior art apparatus for recording electromyograms, i.e., electromyographs (EMG's), record electrical signals taken from the surface of the skin external to the muscles using a pair of surface electrodes. When analyzing a patient, each of the major muscle groups in the legs is measured. To accomplish this analysis, it is necessary to measure and record at least four pairs of muscle signals from each leg, and from the opposing muscles in the calf, thigh, and hips. Prior art devices have typically provided each muscle signal to a common amplifier/transmitter using a thin flexible two-wire cable that transmits the measured signal from each of the surface electrodes to a data acquisition pack carried by the patient. These prior art data acquisition packs receive each of the signals from the electrodes, multiplex the signals into one multiplexed signal, and send a single radio signal from the transmitter carried by the patient to a centralized receiver. The receiver receives the multiplexed signal, demultiplexes the channels, and feeds the individual EMG measurement signals to a data analysis computer.

As shown in FIG. 4, current EMG systems used in gait analysis laboratories are bulky, heavy, and cumbersome. Such EMG systems have wires connecting electrodes for each muscle to the data acquisition pack. These systems can be troublesome because the wires leading to the data acquisition equipment can inhibit motion. Additionally, these systems tend to be heavy because the data acquisition equipment, which is typically located in a pack on the patient, contains the data conditioning, digitization, and multiplexing circuitry, as well as the transmitter and batteries. The weight of the data acquisition pack and the interconnecting wires can alter typical gait patterns, resulting in misrepresentation during analysis, especially for small children. Further, the wires connecting the electrodes to the data acquisition pack tend to get caught or snagged while the patient is wearing the device, and often become disconnected from the surface electrode or the data acquisition pack thereby requiring the operator to stop the tests to reconnect the wiring. For small children, the data acquisition pack can weigh as much as 10% of the child's weight. This is particularly problematic where the child is already physically impaired. Finally, existing measurement devices with the electrodes and wires often intimidate pediatric patients with some of them being afraid of the systems during use.

In another prior art device known as an electrocardiograph, electrical signals produced by a patient's heart are measured. The electrocardiograph also includes an electrode pair but has a small transmitter circuit board mounted directly on one electrode and a wire running to the another electrode of the pair. The transmitter circuit amplifies and transmits the voltage difference between the electrode pair using the interelectrode wire both as a reference signal wire and as the transmitter antenna.

### SUMMARY OF THE INVENTION

The invention includes a transmitter and a receiver for telemetering a signal representing a physical response via a telecommunications medium. Each matched transmitter and receiver telemeters one signal received from a monitoring electrode. Multiple transmitter-receiver pairs may telemeter signals in connection with an electromyograph (EMG).

The invention incorporates improved electronics into EMG equipment and methods using commercially available miniature equipment. The invention is small, light, and lacks the traditional wires connecting the monitoring electrodes to the data acquisition and transmitter pack. The present invention produces a series of single, non-multiplexed signals for telemetry. The projected low cost of a device embodying the invention enables a medical testing lab to have multiple backup units for redundancy.

In accordance with one aspect of the invention, a system for telemetering at least one signal representing a physical response includes a transmitter and a receiver for each signal. The transmitter transmits the signal at a selected frequency. The receiver receives the signal at the selected frequency. The selected frequency is unique for each transmitter and receiver pair. In one embodiment, the selected frequency is a radio frequency.

In accordance with another aspect of the invention, a method for measuring a physical response includes transmitting at a selected frequency a signal representing the physical response, and receiving at the selected frequency the transmitted signal. The received signal is recorded on an electromyogram. The selected frequency is unique for each signal recorded on the electromyogram. In one embodiment, the selected frequency is a radio frequency.

Alternatively, the invention may comprise various other methods and apparatuses. Other features will be in part apparent and in part pointed out hereinafter.

### BRIEF DESCRIPTION OF THE DRAWINGS

FIG. 1 is a block diagram of one embodiment of the method and system of the invention illustrating a transmitter and a receiver communicating via a telecommunications medium.

FIG. 2 is a block diagram of one embodiment of the method and system of the invention illustrating a transmitter connected to two electrodes.

FIG. 3 is a flow chart of one embodiment of the method and system of the invention illustrating a transmitter transmitting a signal to a receiver for analysis.

FIG. 4 is a photograph of an existing prior art EMG measurement unit.

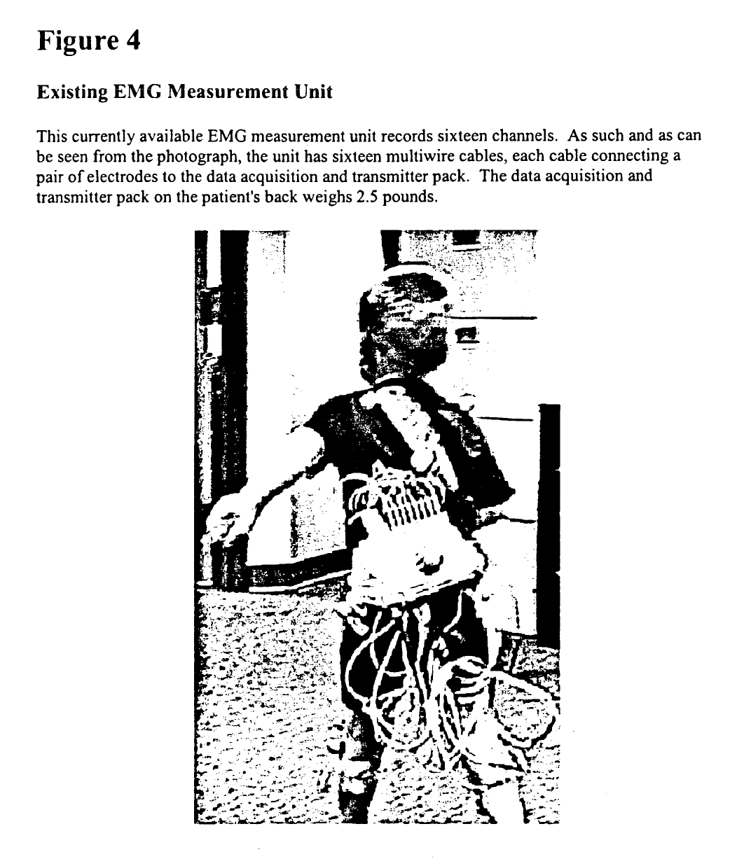

FIG. 5 are graphs showing (a) a raw amplified signal from a “mini” unit, (b) a signal from a Noraxon commercial system, and (c) a filtered signal from a “mini” unit.

FIG. 6 are graphs showing spectrum analyses of (a) a raw amplified signal from a “mini” unit, (b) a signal from a Noraxon commercial system, and (c) a filtered signal from a “mini” unit.

Corresponding reference characters indicate corresponding parts throughout the drawings.

### DETAILED DESCRIPTION OF THE INVENTION

Electromyography is the measurement of the electrical field generated by active muscles. Muscles contract in response to signals from the controlling nerves and the resulting chemical energy exchange across the cell membranes causes ions to move. These moving ions are charged particles that form an electrical current, which in turn generates an electrical field extending through space. When many muscle cells fire continuously, causing the muscle to exert force, the summation of their electrical fields may be measured either nearby or at the surface of the skin directly external to the muscle. Apparatus called electromyographs (EMG's) are used to measure these electrical fields. The measured electrical signal from the muscle may be recorded as an electromyogram.

EMG's have been used to study the on-off timing patterns of muscles during physical actions. An example is gait analysis, in which some of the muscles of the legs are monitored and analyzed during the act of walking to elucidate abnormal patterns. EMG's are used extensively in ergonomic studies to determine when and how dynamic tasks performed by people in certain occupations cause fatigue. EMG's have also been studied to examine relationships between the electrical signals produced by muscles and the forces exerted by a muscle. EMG's are typically used in conjunction with kinematic and kinetic analyses.

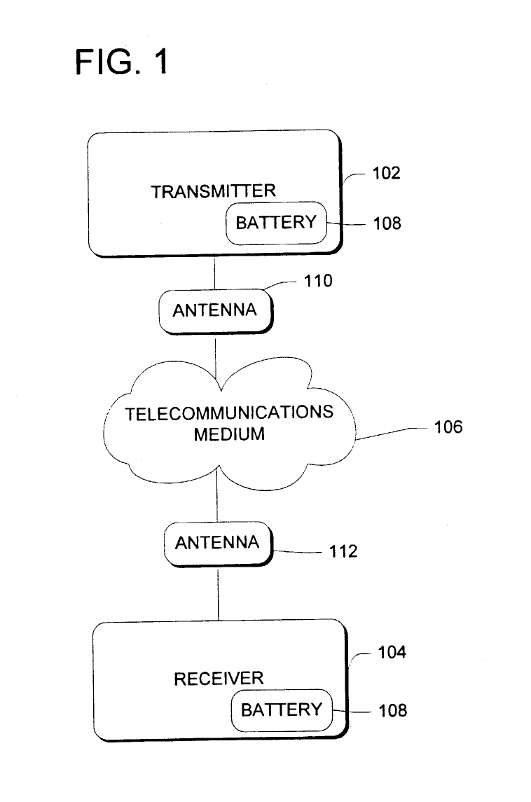

Referring first to FIG. 1, a block diagram illustrates a transmitter 102 and a receiver 104 communicating over a telecommunications medium 106 to telemeter at least one signal representing a physical response. The transmitter 102 transmits a signal at a selected telecommunications frequency from an antenna 110 through a telecommunications medium 106 to a receiving antenna 112 that communicates the received signal to the receiver 104. The receiver 104 receives the signal at the selected telecommunications frequency. The selected telecommunications frequency is unique for each matched transmitter 102 and receiver 104 pair. In one embodiment, the selected telecommunications frequency is a radio frequency. Each receiver 104 and each transmitter 102 include a battery 108. In one embodiment, the battery 108 is rechargeable.

A commercially available transmitter can be modified for use in an EMG to transmit EMG to a modified version of a commercially available receiver. The receiver is modified to pick up the unique frequency, filter the signal for optimum response, and amplify it to ±5 volts, a typical level for digitization.

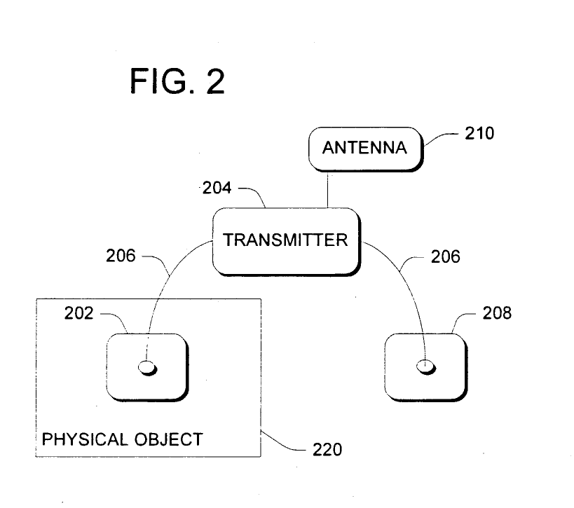

Referring next to FIG. 2, a block diagram illustrates a transmitter 204 (e.g., an FM radio transmitter) connected to a first electrode 202 and a second electrode 208. For an EMG, the first electrode 202 is a signal electrode and the second electrode 208 is a reference electrode. A transmitter 204 is connected to at least one of the electrodes 202, 208. In other embodiments, as shown in FIG. 2, the transmitter 204 is connected to both the electrodes 202, 208. A wire 206 connects the transmitter 204 to the reference electrode 208 and the signal electrode 202. The signal electrode 202 is attached to a physical object, such as human skin overlying a human muscle. In one embodiment, the transmitter 204 includes surface mount components.

Those skilled in the art will note the method and system of the invention are operable using various electrodes. One type of electrode is an indwelling electrode, an electrode that is implanted into the body of the patient. These electrodes include a very fine wire that is inserted directly into the active muscles. Another type of electrode is a surface electrode. Surface electrodes are noninvasive pads that are placed on the surface of the skin directly over a main portion of the muscle. Most gait analysis labs use surface electrodes rather than indwelling electrodes due to their non-invasive nature. Surface electrodes are typically oval or circular and have a radius of about 1 to about 1½ inches. The surface electrodes have chemically treated surfaces, typically containing a gel of sodium chloride that penetrates the skin surface and provides a conductive path for electrical signals from the skin to the transmitter 204.

The present invention includes separate transmitters 102 (FIG. 1) and receivers 104 (FIG. 1) for each muscle measured, thereby enabling placement of the transmitter 204 directly on the signal electrode 202. Placing the transmitter 204 on the signal electrode 202 is an improvement over the prior art because it eliminates wires typically interconnecting each of the electrodes to the data acquisition packs.

In one embodiment, the thickness of the transmitter 204 is about 2 to about 3 mm. This small size allows the patient to sit on or lie in a position where their body is physically on the signal electrode 202 and the transmitter 204 without any adverse effects to the system or the patient. The transmitter 204 is miniaturized by the use of surface mount technology.

In one embodiment, a high input impedance transistor raises the EMG signal amplitude from the electrodes 202, 208 (e.g., about 0.01 to about 5 mV) to a level adequate to modulate the input stage of the transmitter 204 (e.g., about 0.1 V). In one embodiment, the high input impedance transistor also provides an input impedance greater than 10 megaohms, necessary to capture signals from the commercial dry electrodes 202, 208. The EMG signal produces the same percentage frequency modulation as normal voice transmissions, thus generating normal frequency deviations, and allowing a standard FM receiver to demodulate the signal without error.

In one embodiment, the fundamental frequency of a transmitter 204 is determined by the values of the coil and capacitor (or resistor and capacitor) in the oscillator section. The transmitter 204 can exhibit changes in fundamental frequency due to changes of humidity, temperature, voltage, or any of a number of environmental shifts. The transmitter 204 of the invention is intended for the transmission of voice over the standard FM broadcast band. The low cost of the unit and the intended use do not require extreme frequency stability. In one embodiment where frequency stability is necessary (e.g., for low sensor count applications), a crystal controlled design can be used rather than coil-tuned or capacitor-tuned circuits. For example, a crystal controlled oscillator such as the FM-6 commercially available from Ramsey Electronics, Inc. may be used to assure adequate stability. In crystal-controlled embodiments, the frequency determining circuit is tightly controlled by a quartz crystal, which is very resistant to environmental changes. The use of crystals increases the cost of each transmitter 204 and makes them slightly more susceptible to physical impacts. Additionally, in some embodiments, the transmitter 204 circuitry may require a more extensive ground plane to minimize pickup of interfering signals.

The transmitting antenna 210 includes a shield (not shown) over the signal electrode wire 206. Circuit design techniques allow the shield to be grounded at low (audio) frequencies to shield the signal wire 206, while at the same time isolating it from ground at high (radio) frequencies to permit it to also function as an antenna. The antenna 210 may be affected by proximity to the patient's body. Not only is the radio signal attenuated by body fluids, but also placement and body movement causes the antenna electromagnetic field to see an erratic ground plane, which affects frequency stability. Since the antenna 210 and its surroundings also affect transmission range, the antenna 210 provides a good signal to the receivers but not enough to cause interference at great distances. Those skilled in the art will note different sizes and shapes of antennas may be used in the present invention.

In one embodiment, the transmitter 204 includes one watch battery rated at about 1.5 volts and having small current capacity.

A small FM receiver 104 receives the transmitted signal from the transmission medium 106 (FIG. 1) as transmitted by the transmitter 102. One commercially available receiver unit from Ramsey Electronics is the FR-1 receiver, which has a wide tuning range. A wide tuning range is convenient when dealing with multiple operating frequencies. The receiver 104 is constructed on one circuit board for use with other receivers in a multichannel enclosure. Some features such as volume control or tone control are redundant for a unit intended to feed a bandlimited signal of typically about ±5 volts to an analog-to-digital converter board.

The filters in the audio output section of a commercially available receiver are modified to pass the EMG frequency band of about 20 to about 500 Hz. A receiver 104 according to the invention does not require stereo demodulation circuitry. The output amplifier has a nominal about ±5 volts output. A switch allows the output's nominal voltage to be switched from about ±5 volts to about ±1, 5 or 10 volts. First or second order filters are implemented at each stage of amplification to suitably filter and amplitude scale the demodulated output so it is consistent with standard EMG frequency response and to be compatible with existing digitizing equipment. A convenient option includes colored light emitting diodes to indicate high or low signal strength for each transmitter receiver pair.

According to the invention, and in a manner similar to the transmitters 102, the receivers 104 may include coil and capacitor frequency tuning and/or crystal controlled frequency tuning.

In embodiments having a plurality of separate receivers 104, the antenna signal is split among all the receivers 104 in the receiver enclosure. In embodiments where one antenna is insufficient, a powered or “active” antenna may be used. Another embodiment of the invention incorporates more than one antenna 112 spread around the room as a “diversity” reception system.

In one embodiment, a single receiving antenna 112 feeds all the receivers 104 simultaneously and its size determines the reception range. When a transmitter 102 is on a body, the field strength decreases markedly on the side of the body opposite the transmitter. Low signal strength for a particular area of a room may require multiple antennas 112 using diversity reception to feed all the receivers 104 and/or an active or powered antenna 112. In one embodiment, diversity reception may be required to supply signals to eight or more receivers 104.

In an alternative embodiment, a single receiver 104 scans a frequency range to discover and receive signals from all active transmitters 102 within a given area. The single receiver 104 then interfaces with a storage facility to digitize and store all the simultaneously received signals.

In an alternative embodiment, the transmitter 102 and the receiver 104 communicate on a frequency in the Medical/Industrial band (e.g., in the 600 MHz or 800 MHz range).

Electronic devices attached to patients run the risk of being harmed by static electricity discharges especially during dry weather or when patients are on carpets. Input protection against static electricity is incorporated according to standard engineering practices, such as the incorporation of resistors or diodes between the active input leads and ground to “leak” the static charge to ground. The values must be chosen to present an extremely high impedance at the signal frequencies to minimize any signal loss.

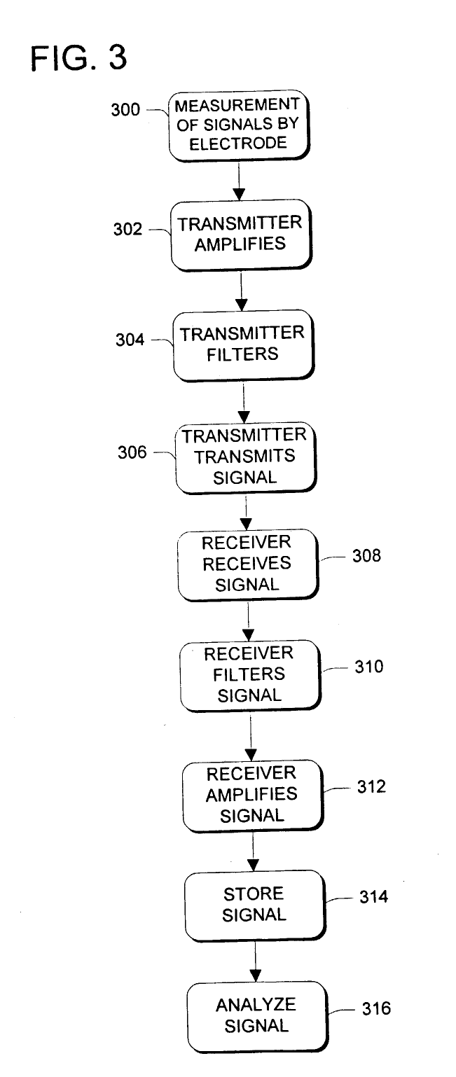

Referring next to FIG. 3, a flow chart illustrates a transmitter 102 (FIG. 1) transmitting a signal to a receiver 104 (FIG. 1) for analysis. An electrode 202, 208 (FIG. 2) measures 300 a signal representing a physical response. The transmitter 102 amplifies 302, filters 304, and transmits 306 the signal to the receiver 104 at a selected frequency. The receiver 104 receives 308 the transmitted signal at the selected frequency. The selected frequency is unique for each signal. In one embodiment, the selected frequency is a radio frequency. The receiver 104 filters 310 the signal for optimum response, and amplifies 312 the signal. In one embodiment, the receiver 104 amplifies the signal to about ±5 volts for digitization. The received signal is stored 314 and analyzed 316.

Once the signals from multiple electrode pairs are presented to the analysis equipment, they may be stored in a computer for later analysis, or they may be printed out for review. The method of analysis depends on the initial reason for data collection. If the investigator is interested in reviewing synchronization of the muscles with physical actions such as walking (gait analysis), then a printout showing the EMG signals and the physical action provides the investigator information about the timing of events.

Computer analysis is often used, since it can measure and analyze the amplitude and other parameters of the electromyogram that are not obvious to the eye. Several methods of calculating the amplitude parameters exist, including rectification, linear envelope, and integration. One common method of looking at the amplitude is called the “Root Mean Square” value, a signal analysis method favored by engineers for a wide variety of amplitude measurements. It consists of squaring the signal, averaging the result, and then taking the square root of that number; a calculation well suited to computers. Another analysis method involves calculating the Fourier Transform to examine the frequency spectrum of the signal. Some parameters related to frequency have been suggested, such as the median frequency, the frequency at which half the spectral power is above the median, and half is below that frequency. The combination of RMS and median frequency has been established as the best measure of muscle fatigue.

In one embodiment, eight units embodying the invention are consolidated to form a single system, with a separate transmitter 102 and receiver 104 for each channel. The eight transmitter units 102 are separate and unique, but the eight receivers 104 may be incorporated into a single enclosure and feed signals to a data acquisition system. The receivers 104 are housed in a common box, with one antenna 112 and with outputs to multiple channels of a data acquisition computer. In some embodiments when the receivers 104 are in close proximity, they may need more extensive shielding to minimize crosstalk. The separate receivers 104 supply the signals to the data acquisition equipment. Adequate shielding and isolation between the receivers 104 reduces crosstalk and the reception of unwanted radio signals. Signal frequency separation permits the operation of multiple units. Use of alternative frequency bands in some embodiments minimizes interference with external radio signals. In one embodiment, each transmitter-receiver pair uses a frequency that is distant from the FM band. More than one receiving antenna 112 may be necessary to pick up signals within a room. The antenna 112 may need to be an active (powered) type if coverage proves to be a problem in large rooms. Another embodiment of the invention accommodates up to 32 channels.

The apparatus and method may be used to form an electromyogram, an electrocardiogram or an electroencephalogram. In one embodiment, the physical object is a human and each signal represents the measured physical response of a single muscle. The muscles include, but are not limited to, the following muscles from the left and right side of a human: tibialis anterior, gastrocnemius, hamstrings, rectus femoris, adductors, gluteus maximus, gluteus medius, and tensor fasciae latae.

The concept of miniaturizing the transmitter 102 is also directly applicable to electroencephalographs, electrocardiographs and stress tests. The activity during a stress test tends to strain the wires and connectors, so elimination of interconnecting and often dangling wires presents fewer problems when performing stress tests. Electrocardiograph telemetry as presently implemented in many hospitals uses transmitter boxes more bulky than the present invention. Inclusion of heart and intensive care unit patients increases the potential population of patients who benefit from less restrictive telemetry packages. There are other possible applications according to the invention for minimally invasive telemetry for use during therapy, rehabilitation, and biofeedback, as well as for patients having spinal cord injury or multiple sclerosis.

In order to demonstrate the advantages of transmitting an EMG signal via telemetry with miniaturized components, a Ramsey FM5 transmitter and other readily available miniaturized components were used to build a “mini” transmitter. The results of the “mini” transmitter were compared to those obtained from an existing commercial Noraxon EMG system equipped with interconnecting wires between the electrodes and the data acquisition pack. In both cases, the two pairs of electrodes were placed as close together as possible over the main portion of the muscle. When the muscle was contracted, the ‘mini’ transmitter sent the signal to a simple FM radio receiver, while the Noraxon unit sent a multiplexed signal to its receiver. The output jack of the FM radio receiver was connected to the same data acquisition equipment as the Noraxon receiver.

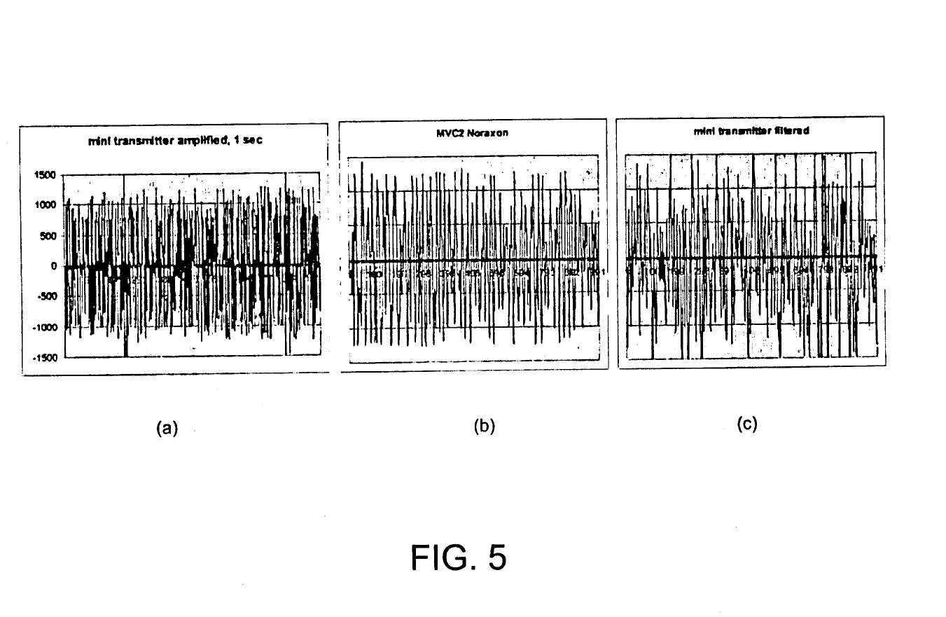

The raw amplified signal from the “mini” unit (FIG. 5a) was compared directly to the raw signal from the Noraxon unit (FIG. 5b). The transmitter and FM receiver were designed for audio frequencies in a range from about 50 Hz to about 15,000 Hz, while the Noraxon unit was designed for EMG frequencies in a range from about 20 Hz to about 130 Hz. Thus, the results from the “mini” unit contain additional signals that were internally filtered with the Noraxon system. When the signal from the “mini” unit was filtered to match the Noraxon bandwidth signal (FIG. 5c), the data were more similar.

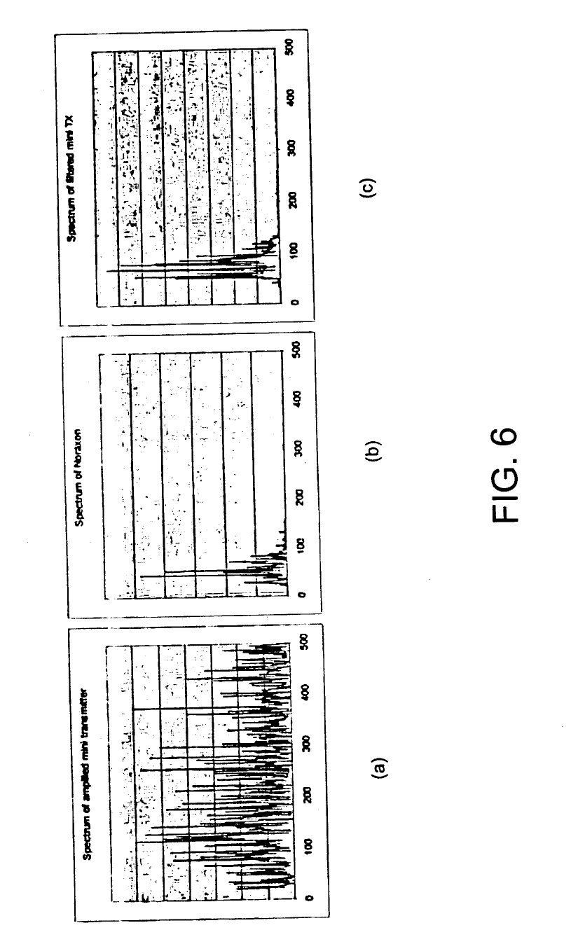

To evaluate the similarities of the signals, the frequency response was analyzed using a Fourier analysis (FIGS. 6a-c). As expected, the unfiltered signal from the “mini” unit (FIG. 6a) contained frequencies over the audio frequency bandwidth (i.e., 50 to 15,000 Hz). Conversely, the signal from the Noraxon unit contained frequencies over its electronically filtered bandwidth (i.e., 20 to 130 Hz) (FIG. 6b). Although the frequency spectrum of the filtered data from the “mini” unit (FIG. 6c) is close to the Noraxon signal, it is still not identical. This was primarily due to the design specifications of the FM radio receiver that amplified the higher frequency audio signals and minimized the lower frequency non-audio signal. Of course, one skilled in the art would recognize that a 20 Hz signal cannot be heard by most human ears. A bass drum or an organ can produce an audible sound slightly below 50 Hz. A mans voice is in the range of 100-200 Hz, and a woman's from 200-400 Hz.

These results demonstrate the advantages of the present system and method. With components designed specifically to match the output of commercial EMG systems (e.g., to limit the signal frequency to acceptable EMG norms), the present system and method produce results comparable to the commercial systems.

Additional tests will be conducted as described in Appendix A. These include technical testing, environmental testing of the transmitters and receivers, ergonomic testing, and human testing.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As various changes could be made in the above products and methods without departing from the scope of the invention, it is intended that all matter contained in the above description and shown in the accompanying drawings shall be interpreted as illustrative and not in a limiting sense.

1. A system for measuring electrical signals produced by tissue of a patient comprising:
a plurality of electrodes adapted for attachment to selected sites on the patient corresponding to tissue of interest;
a plurality of transmitters, each transmitter of said plurality of transmitters being connected to at least one electrode of said plurality of electrodes and adapted to transmit a signal at a telecommunications frequency uniquely selected from a plurality of telecommunications frequencies to correspond to the electrode of said plurality of electrodes to which the transmitter is connected; and
a receiver configured to receive at least one signal transmitted by said plurality of transmitters at the corresponding uniquely selected telecommunications frequency.

2. A system as set forth in claim 1 wherein each transmitter of said plurality of transmitters is connected to a reference electrode and a signal electrode.

3. A system as set forth in claim 1 wherein the system comprises a plurality of receivers including aforesaid receiver, each receiver of said plurality receivers being configured to receive a transmitted signal from at least one of said plurality of transmitters.

4 A system as set forth in claim 1 wherein the plurality of electrodes are selected from the group consisting of: an electromyograph sensor; an electrocardiograph sensor; and an electroencephalograph sensor.

5. A system as set forth in claim 1 further comprising a rechargeable battery operatively connected to each receiver and each transmitter.

6. A system for telemetering a plurality of signals representing physical response of a patient, said system comprising:
a plurality of transmitters, each transmitter of said plurality of transmitters being adapted to transmit at least one signal of said plurality of signals at a telecommunications frequency uniquely selected from a plurality of telecommunications frequencies to correspond to the signal which the transmitter is adapted to transmit; and
a receiver for receiving the signal transmitted by at least one transmitter of said plurality of transmitters at the corresponding uniquely selected telecommunications frequency.

7. A system as set forth in claim 6 wherein the receiver is adapted for receiving each signal transmitted by said plurality of transmitters at the corresponding uniquely selected telecommunications frequencies.

8. A system as set forth in claim 6 wherein the plurality of signals are produced by an apparatus selected from a group of apparatus consisting of: an electromyograph; an electrocardiograph; and an electroencephalograph.

9. A system as set forth in claim 6 wherein each transmitter of said plurality of transmitters is connected to at least one electrode.

10. A system as set forth in claim 9 wherein each transmitter of said plurality of transmitters is connected to a reference electrode and a signal electrode.

11. A system as set forth in claim 10 further comprising at least one wire connecting each transmitter of said plurality of transmitters to the corresponding reference electrode and the corresponding signal electrode.

12. A system as set forth in claim 9 wherein each electrode is adapted for connection to a human patient and each signal represents an electrical charge output by a muscle.

13. A method for measuring a physical response comprising the steps of:
transmitting a signal representing the physical response at a radio frequency uniquely selected from a plurality of radio frequencies to correspond to the signal being transmitted; and
receiving the transmitted signal.

14. A method as set forth in claim 13 further comprising the step of storing the received signal.

15. A method as set forth in claim 14 further comprising the step of analyzing the stored signal.

16. A method as set forth in claim 13 further comprising the steps of amplifying and filtering the signal before transmission.

17. A method as set forth in claim 13 further comprising the step of amplifying the received signal to ±5 volts for digitization.
